(19) United States
(12) Patent Application Publication
(10) Pub. No.: US 2023/0012444 A1
(43) Pub. Date: Jan. 12, 2023

(54) A Drug Delivery Device

(71) Applicant: Sanofi, Paris (FR)

(72) Inventors: Oliver Charles Gazeley, Warwick, Warwickshire (GB); Craig Ashley Mason, Warwick, Warwickshire (GB); Richard James Thomas, Warwick, Warwickshire (GB)

(21) Appl. No.: 17/781,940

(22) PCT Filed: Dec. 8, 2020
(86) PCT No.: PCT/EP2020/084972

§ 371 Date: Jun. 2, 2022

(30) Foreign Application Priority Data
Dec. 9, 2019 (EP) 19306600.8

(51) Int. Cl. A61M 5/315

(52) U.S. Cl. CPC A61M 5/31591; A61M 5/31551; A61M 5/31568; A61M 2205/52; A61M 2205/581

(57) ABSTRACT

A drug delivery device includes a housing, a drug delivery mechanism within the housing and displaceable between pre-delivery and mechanical stop positions, and an end of dose switch. The end of dose switch includes a first sensor disposed on the drug delivery mechanism and a second sensor element disposed within the housing. The switch is configured to be triggered when relative movement of the first and second sensors causes the first and second sensor elements to move into a switch position. The delivery mechanism is biased in a proximal direction away from the mechanical stop position. The device includes a controller configured to: record when the end of dose switch is triggered;

measure a duration of time between triggering the switch and the first and second sensors moving out of the switch position; and generate an error code indicative of a deviation between manipulations applied and a predetermined handling scheme.

### CROSS REFERENCE TO RELATED APPLICATIONS

The present application is the national stage entry of International Patent Application No. PCT/EP2020/084972, filed on Dec. 8, 2020, and claims priority to Application No. EP 19306600.8, filed on Dec. 9, 2019, the disclosures of which are incorporated herein by reference.

### TECHNICAL FIELD

The present disclosure relates to a drug delivery device.

### BACKGROUND

Pen-type drug delivery devices are useful for regular injection by persons without formal medical training. This is increasingly common among patients having diabetes where self-treatment enables such patients to conduct effective management of their diabetes. For good or perfect glycemic control, the dose of insulin or insulin glargine has to be adjusted for each individual to achieve a desirable blood glucose level.

### SUMMARY

The present disclosure relates to injectors, for example handheld injectors, especially pen-type injectors. The present disclosure relates to injectors that provide for administration by injection of medicinal products from a multidose cartridge. In particular, the present disclosure relates to such injectors where a user may set the dose. The dose to be injected may be, for example manually selected at the injector by turning a dosage knob and observing the actual dose from a dose window or display of the injector device.

A user undertaking self-administration of insulin will commonly need to administer between 1 and 80 International Units. To monitor dosages for preventing false handling of the device or keeping track of the doses already applied, it is desirable to measure information related to a condition and/or use of the injection device, such as, for example, information on the injected dose.

According to the fundamentals of the present disclosure, there is provided a drug delivery device comprising: a housing having a proximal end and a distal end; a drug delivery mechanism provided at least partially within the housing and displaceable between a pre-delivery position and a mechanical stop position to dispense medicament from the drug delivery device when a user presses down on a proximal end of the drug delivery mechanism; and an end of dose switch configured to be triggered prior to the drug delivery mechanism reaching the mechanical stop position, the end of dose switch comprising a first sensor element disposed on the drug delivery mechanism and a second sensor element disposed within the housing;

wherein the end of dose switch is configured to be triggered when relative movement of the first sensor element and the second sensor element causes the first and second sensor elements to move into a switch position, and wherein the drug delivery mechanism is biased in a proximal direction away from the mechanical stop position, so that, when a user ceases to press on the proximal end of the drug delivery mechanism, the first and second sensor elements move out of the switch position;

the drug delivery device further comprising a controller configured to:

record when the end of dose switch is triggered; measure a duration of time between the end of dose switch being triggered and the first and second sensor elements moving out of the switch position; and generate an error code indicative of incorrect use of the device when the measured duration is less than a predetermined time period.

The drug delivery mechanism may be displaceable relative to the housing between a pre-delivery position and a mechanical stop position to dispense medicament from the drug delivery device.

Said error code may further indicate that the dose of medicament delivered during use of the device is less than a predetermined dose of medicament.

Therefore, if the user incorrectly uses the device, said incorrect use is recorded by the generation of an error code.

The error code may alert the user to the possibility that the dose of medicament delivered during use of the device is less than the predetermined dose of medicament.

The switch position may be any position of the first and second sensor elements that causes the end of dose switch to be triggered.

The first and second sensor elements may move into the switch position when the second sensor element moves over a distal edge of the first sensor element.

Therefore, the switch position may be established by a simple sensor arrangement.

The end of dose switch is configured to be triggered when the first sensor element moves in a distal direction over the switch position 340.

Therefore, in embodiments of the disclosure, relative movement of the first sensor element over the second sensor element may be used to generate a signal.

The first sensor element may comprise a conductive strip on the drug delivery mechanism that is electrically connected to the controller.

Therefore, a signal may be generated when the first sensor element makes contact with the second sensor element.

The second sensor element may comprise a bridging contact configured to connect the conductive strip to a live strip on the drug delivery mechanism to cause an electrical signal to be transmitted to the controller.

The bridging contact may be formed as a metal pressing and comprise contact points disposed at ends of cantilevered members of the metal pressing, said contact points being configured to contact the live strip and the first sensor element respectively to electrically connect the live strip with the sensor element.

The second sensor element may comprise two or more bridging contacts spaced around an inner surface of the housing.

Therefore at least one live strip is connected to at least one conductive strip irrespective of a rotational position of the drug delivery mechanism with respect to the housing.

The two or more bridging contacts may be provided at the proximal end of the housing.

The drug delivery mechanism may comprise a dose setting component, the dose setting component being rotatable relative to the housing to set a drug dose to be administered; and wherein the first sensor element comprises a series of conductive strips spaced around an outer surface of the dose setting component.

Therefore, the first sensor element may be used as part of a rotary encoder to encode the drug dose set by the dose setting component.

Said dose setting component may further comprise conductive dose encoding strips and live strips alternately spaced around the outer surface of the dose setting component, the dose encoding strips each being electrically connected to the controller, and wherein the bridging contacts connect and disconnect the dose encoding strips to the live strips when the dose setting component rotates relative to the housing to transmit electrical signals to the controller and encode the set drug dose.

The first sensor element may comprise a plurality of conductive strips spaced around the proximal end of the dose setting component.

Therefore, the end of dose switch is triggered just prior to drug delivery mechanism moving into the mechanical stop position.

The plurality of conductive strips of the first sensor element may be spaced apart to allow the dose encoding strips to extend in between and into electrical connection with the controller.

The conductive strips and/or dose encoding strips may be printed, plated or etched onto an exterior surface of the dose setting component.

The controller may be configured to record the encoded set drug dose when the end of dose switch is triggered.

The controller may be provided at a proximal end of the dose setting component so that a compact device is provided.

The predetermined time period may be based on human reaction time.

This ensures that for the vast majority of cases, the dose will be logged as being fully dispensed before a user can react to reaching the mechanical stop and ceases to press on the drug delivery mechanism.

The predetermined time period may be between 0.2 s and 0.4 s.

The predetermined time period is preferably 0.25 s.

The drug delivery device may be configured to provide a visual and/or audio cue to a user of the drug delivery device to signal that a set drug dose has been administered following expiration of the predetermined time period.

Therefore, the user is informed that they may cease to press on the drug delivery mechanism.

The device may be configured to provide a visual and/or audio cue to a user of the device to warn a user that a set drug dose may not have been completely administered when the user ceases to press on the proximal end of the drug delivery mechanism prior to the end of the predetermined time period.

### BRIEF DESCRIPTION OF THE FIGURES

The following description is with reference to the following Figures:

FIG. 1 shows an external view of a drug delivery device;

FIG. 2 shows a schematic diagram of electronic components present in the drug delivery device of FIG. 1;

FIGS. 3A and 3B show perspective views of part of a drug delivery mechanism of a drug delivery device;

FIGS. 4A and 4B show a plan view of part of a drug delivery mechanism of a drug delivery device;

FIG. 5 shows an example conductive strip arrangement;

FIG. 6 shows an example conductive strip arrangement;

FIG. 7 is a schematic diagram of an end of dose switch;

FIG. 8 shows a bridging contact;

FIGS. 9A and 9B show a dose delivery button in an out condition and an in condition, respectively;

FIG. 10 is a schematic diagram showing a switch position 340 relative to a mechanical stop position;

FIG. 11 is a schematic diagram showing an order of events of a user operating a drug delivery device;

FIG. 12 is a flow chart showing three outcomes of a first use case scenario;

FIG. 13 is a flow chart showing two outcomes of a second use case scenario;

FIG. 14 is a schematic diagram showing an end of dose switch; and

FIG. 15 is a schematic diagram showing an end of dose switch.

### DETAILED DESCRIPTION

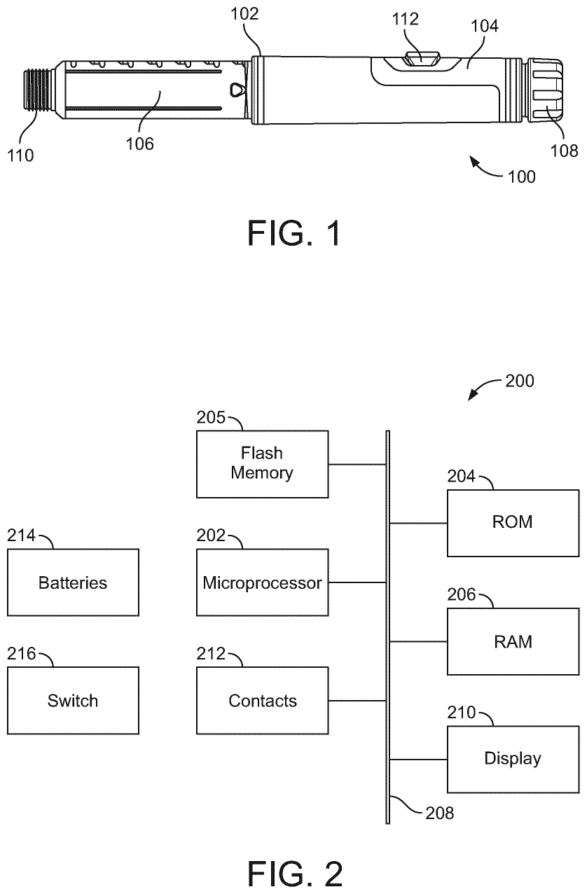

Referring firstly to FIG. 1, an external view of a drug delivery device 100 according to embodiments of the disclosed subject matter is shown. The device 100 shown in FIG. 1 is a pen type injection device, having an elongate cylindrical shape, for setting and delivering a medicament, such as insulin. The device 100 comprises a housing 102 having a first housing part 104 and a second housing part 106. A rotatable dial 108 is located at a first (or proximal) end of the first housing part 104. The rotatable dial 108 has substantially the same outer diameter as the first housing part 104. The second housing part 106 may be detachably connected to the second end of the first housing part 104. The second housing part 106 is configured to have a needle (not shown) or similar drug delivery apparatus attached to it. To achieve this, the second (or distal) end of the second housing part 106 may have a threaded portion 110. The threaded portion 110 may have a smaller diameter than the remainder of the second housing part 106.

A display mount 112 is located on the first housing part 104. A display 210 may be supported on the display mount 112. The display 210 may be an LCD display, a segmented display or any other suitable type of display. The display mount 112 may cover a recess (not shown) in the first housing portion 104. A number of electronic components, described in greater detail with reference to FIG. 2, may be disposed underneath the display mount 112.

The first housing part 104 contains a drug dose setting and delivery mechanism. The second housing part 106 contains a drug cartridge (not shown). The drug contained in the drug cartridge may be a medicament of any kind and may preferably be in a liquid form. The drug delivery mechanism of the first housing part 104 may be configured to engage with the drug cartridge of the second housing part 106 to facilitate expulsion of the drug.

The second housing part 106 may be detached from the first housing part 104 in order to insert a drug cartridge or to remove a used cartridge. The first and second housing parts 104, 106 may be connected to each other in any suitable way, for example with a screw or bayonet type connection. The first and second housing parts 104, 106 may be non-reversibly connected to each other in such a way that the drug cartridge is permanently contained within the drug delivery device 100. Further the first and second housing parts 104, 106 may form part of a single housing part.

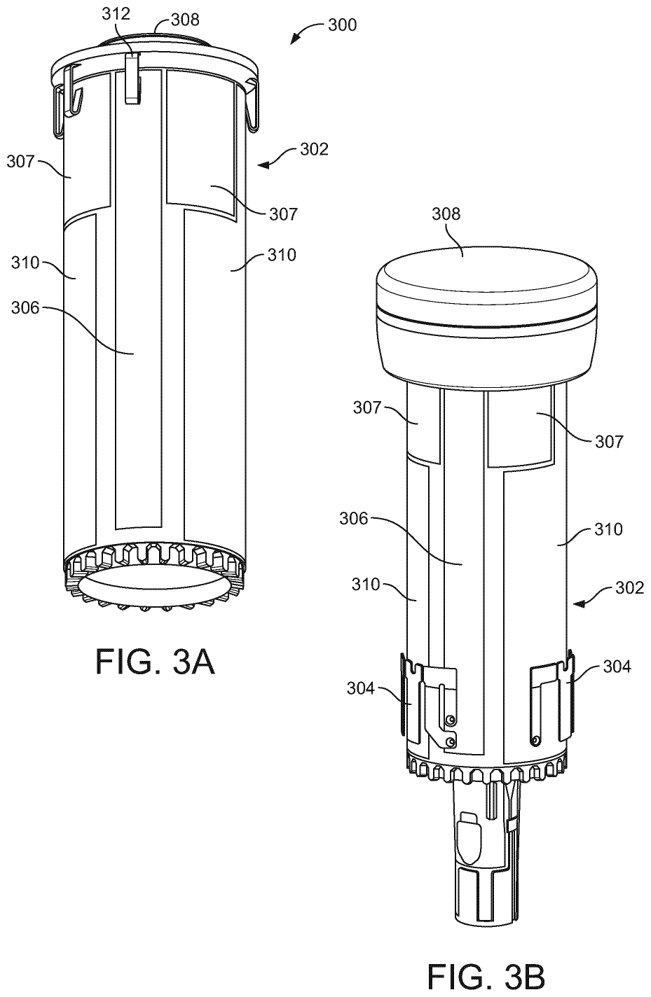

The rotatable dial 108 is configured to be rotated by hand by a user of the drug delivery device 100 in order to set a drug dose to be delivered. The dial 108 may be connected to a movable dosage programming component (302 in FIG. 3) of the dose setting mechanism (300 in FIG. 3) comprising an internal threading system which causes the dial 108 to be displaced axially from the housing 102 as it is rotated in a first direction. The dial 108 may be rotatable in both directions or only in a first direction. The device 100 is configured, once a drug dose has been set by rotation of the rotatable dial 108, to deliver the set drug dose when a user exerts an axial force at the proximal end of the device. The rotatable dial 108 may support a dose delivery button (308 in FIG. 3) which must be depressed in order to deliver the set drug dose. The display 210 may be configured to display information concerning the drug dose which has been set and/or delivered. The display 210 may further show additional information, such as the actual time, the time of the last usage/injection, a remaining battery capacity, one or more warning signs indicating that a dialled dose has not been fully dispensed, and/or the like.

Referring now to FIG. 2, a schematic diagram of electrical circuitry 200 forming part of the drug delivery device 100 is shown. The circuitry 200 comprises a controller 202 (herein processor 202), a non-volatile memory such as a ROM 204, a writable non-volatile memory such as flash memory 205, a volatile memory such as a RAM 206, the display 210, conductive strips 212 and a bus 208 connecting each of these components. The circuitry 200 also comprises batteries 214 or some other suitable source of power for providing power to each of the components and a switch 216, described in greater detail below.

The circuitry 200 may be integral with the device 100. Alternatively, the circuitry 200 may be contained within an electronic module that can be attached to the device 100. In addition, the circuitry 200 may comprise additional sensors, such as optical or acoustical sensors. The circuitry 200 may comprise an audible alarm (not shown) which the processor 202 may control to sound an alarm when a dialled dose has not been fully dispensed.

The ROM 204 may be configured to store software and/or firmware. This software/firmware may control operations of the processor 202. The processor 202 utilises RAM 206 to execute the software/firmware stored in the ROM to control operation of the display 210. As such the processor 202 may also comprise a display driver. The processor 202 utilises the flash memory 205 to store determined amounts of dose dialled and/or determined amounts of dose dispensed, as will be described in more detail below. The processor 202 may be a microcontroller or microcontroller unit.

The batteries 214 may provide power for each of the components including the conductive strips, 212. The supply of electricity to the conductive strips 212 may be controlled by the processor 202. The processor 202 may receive signals from the conductive strips 212 and so could determine when the conductive strips 212 are energised and is configured to interpret these signals. Information may be provided on the display 210 at suitable times by operation of the software/firmware and the processor 202. This information may include measurements determined from the signals received by the processor 202 from the conductive strips 212.

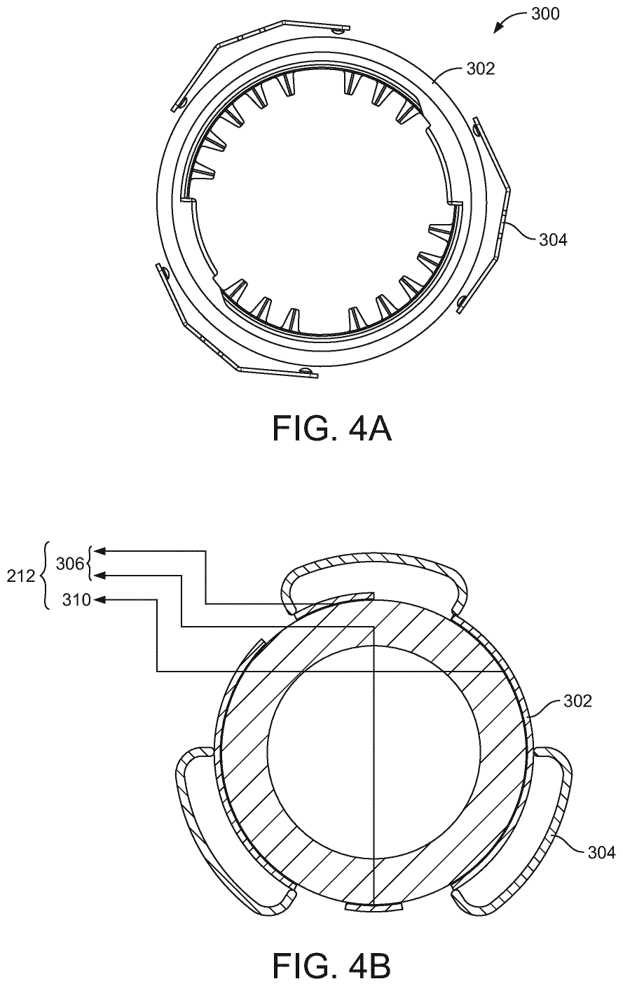

A fuller explanation of the operation of the dose setting mechanism 300 will now be given with reference to FIGS. 3 to 6. FIGS. 3A and 3B show perspective views of part of a dose setting mechanism 300 of a drug delivery device 100 suitable for use with the disclosure, and FIGS. 4A and 4B show a plan view of part of a dose setting mechanism 300 of a drug delivery device 100 suitable for use with the disclosure.

The movable dosage programming component 302 of this embodiment is cylindrical and is arranged to rotate relative to the first part 104 of the housing 102 during programming of a dosage (but does not rotate relative to the housing 102 during delivery of said dose). The rotation of this component is encoded by selectively connecting and disconnecting conductive strips 212 on the dosage programming component, thereby alternating electrical signals received by processor 202. Processor 202 may be implemented within any suitable electronic module containing electrical circuitry 200.

The electronic module containing circuitry 200 may be embedded within button 308, which can eliminate the requirement to remove and re-use the electronic module when being used in conjunction with a disposable pen injector or other disposable drug delivery device. The embedded electronic module can enable the recording of doses that are dialled and delivered from the pen. This functionality may be of value to a wide variety of device users as a memory aid or to support detailed logging of dose history. It is envisaged that the electronic module could be configured to be connectable to a mobile device, or similar, to enable the dose history to be downloaded from the module on a periodic basis.

As described with reference to FIGS. 3A and 3B, the dosage programming component 302 may comprise a number of the conductive strips 212. The conductive strips may be printed, plated, or etched on an exterior surface of the movable dosage programming component 302 (which exterior surface may be contained within the housing 102 when no dosage as set, as in the arrangement shown in FIG. 1). For example, the conductive strips 212 may be formed from conductive ink. Some of the conductive strips 212 are live “source” strips 310, which are electrically connected to a voltage supply to provide an electrical potential. Other of the conductive strips are “sensor” strips 306, which electrically connected to input terminals of the processor 202. In this embodiment, the conductive strips 212 are electrically connected to the electrical circuitry 200 embedded in the button 308 by metallic contacts 312, but any other suitable electrical contact may be provided.

Bridging contacts 304 electrically connect two of the conductive strips 212. The bridging contacts 304 are mounted within the first part 104 of the housing 102 and are not electrically connected to processor 202. Rather, the bridging contacts 304 act to provide a conductive path between two of the conductive strips 212. In one embodiment of the bridging contacts 304, they are formed using a metal pressing (using stainless steel, for example), with three contact points 304a-304c formed as bumps, as shown in FIG. 8. This manufacturing approach may facilitate the provision of a low cost bridging contact. The contact points 304a-304c are formed at the end of cantilevered members to allow a pre-load to be achieved, ensuring good radial contact pressure with the conductive strips 212, even in worst case tolerance conditions. The bridging contacts are here rotationally and axially aligned within the cylindrical housing 102.

In this embodiment, the bridging contacts 304 alternately couple and de-couple source strips 310 to adjacent sensor strips 306 as the dosage programming component 302 rotates (illustrated in FIG. 4B). If the sensor strip 306 is connected via a bridging contact 304 to a live source strip 310, a voltage state of the sensor strip 306 is considered to be ‘1’, otherwise the voltage state is ‘0’. By using the voltage state of the sensor strips 306 as an input to processor 202, it is possible to generate a Gray code describing increments of rotational position, and the direction of rotation.

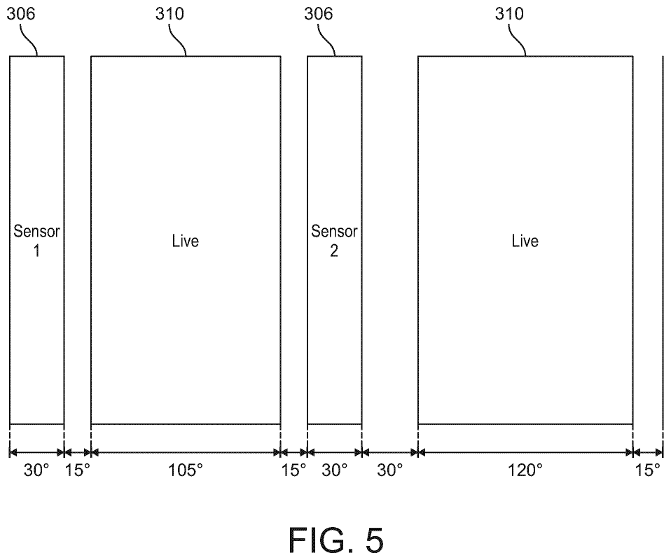

FIGS. 3 and 4 illustrate an embodiment comprising four vertical conductive strips 212 (two live source strips 310 and two sensor strips 306, alternately arranged), which is suitable for encoding 24 units of dosage. Rather than use a detector to read a code printed on the dosage programming component, this arrangement uses the electrical state of the conductive strips 212 themselves to form an input to the processor 202. In this arrangement, the rotation of the dosage programming component 302 can be encoded electronically to identify the selected dose value before the dose is delivered. The simplest Gray code that can be used to count doses and detect direction of rotation is a 2-bit Gray Code. The embodiment shown above uses three bridging contacts, spaced equidistant around a circumference of the dosage programming component 302, and each bridging between two points on the cylinder that are 60° apart. The conductive pattern (shown in 2D below in FIG. 5) below has a variable strip width and gap ratio, and in conjunction with the three equi-spaced Bridging Contacts as described above, forms a 2-bit quadrature signal during rotation. The black areas represent regions of conductive material, and the white areas represent regions where no conductive material has been deposited. However, there are a number of configurations of conductive strips 212 (number of strips, width of strips, etc.) and bridging contacts 304 that will generate a cyclical Gray code during rotation and so could be used to encode the desired dosage setting.

Once the dose has been encoded in the manner described above, a user may deliver the set drug dose by exerting an axial force to the dose delivery button 308, displacing the drug delivery button 308 toward the proximal end of the first housing part 104. Therefore, the drug delivery mechanism is displaced relative to the housing 102 with the dose delivery button 308 to expel medicament from the drug cartridge. A mechanical stop (320 in FIG. 10) physically prevents further displacement of the drug delivery button 308 when the whole dose has been dispensed. The mechanical stop 320 may comprise part of the housing 102 which is configured to abut part of the drug delivery mechanism or the dial 108.

An end of dose switch is provided to record that the dose has been dispensed. When the switch is triggered the encoded dose is electronically committed to memory. The switch is herein referred to as a ‘zero unit’ switch, or ‘0U’ switch.

In one embodiment, the switch comprises first and second sensor elements 313, 314 disposed on the programming component 302 and within the housing 102, respectively. The end of dose switch is triggered when relative movement of the first and second sensor elements 313, 314 causes the first and second sensor elements 313, 314 to move into a switch position 340. Therefore the switch position 340 is defined as any position of the first and second sensor elements 313, 314 that causes the end of dose switch to be triggered. In one embodiment, the first and second sensor elements 313, 314 move into the switch position 340 when the second sensor element 314 moves over a distal edge 330 of the first sensor element 313. Therefore the switch is triggered when the second sensor element 314 moves over the distal edge 330 of the first sensor element 313, as will be explained further below.

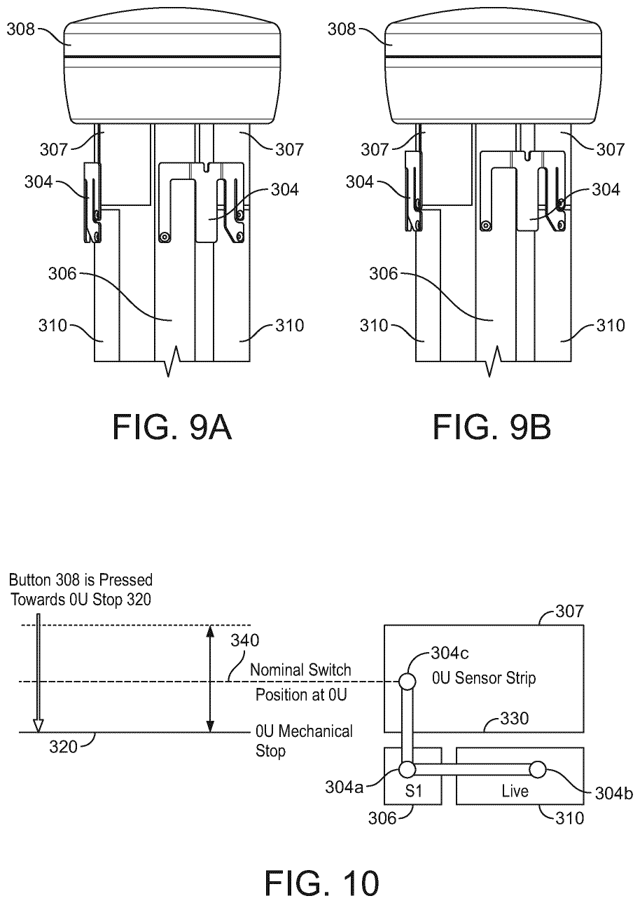

In the embodiment illustrated by FIGS. 3a, 3b, 9a and 9b the first sensor element 313 comprises an additional sensor strip 307 at the proximal end of the programming component 302. The second sensor element 314 consists of the bridging contacts 304 described above.

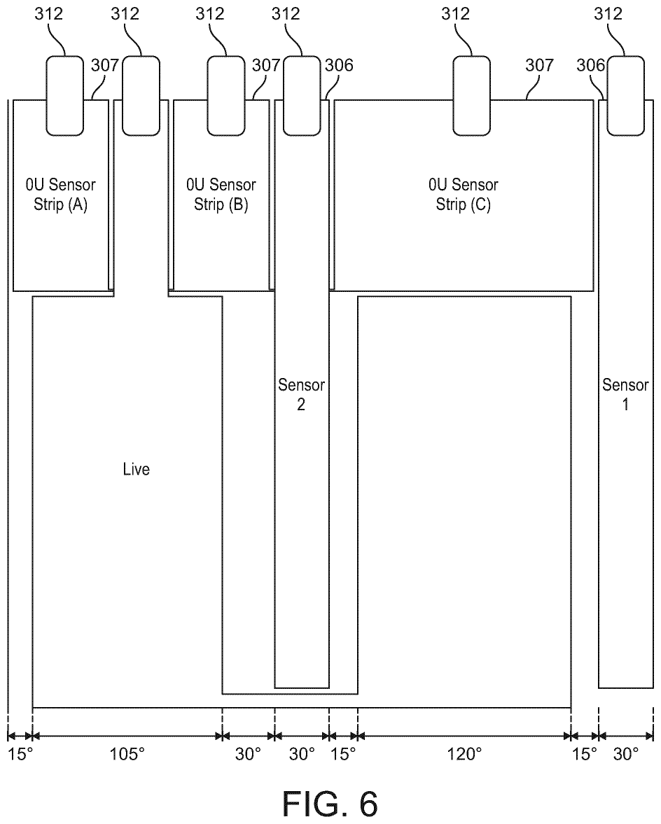

The additional sensor strip is herein referred to as a zero unit sensor strip 307 (0U sensor strip 307). The 0U sensor strip 307 is divided into discreet areas 307a-307c spaced circumferentially about the programming unit 302 to allow other live 310 and sensor strips 306 to pass in between for electrical connection with metallic contacts 312. The conductive pattern including the 0U sensor strips 307a-307c is shown in FIG. 6.

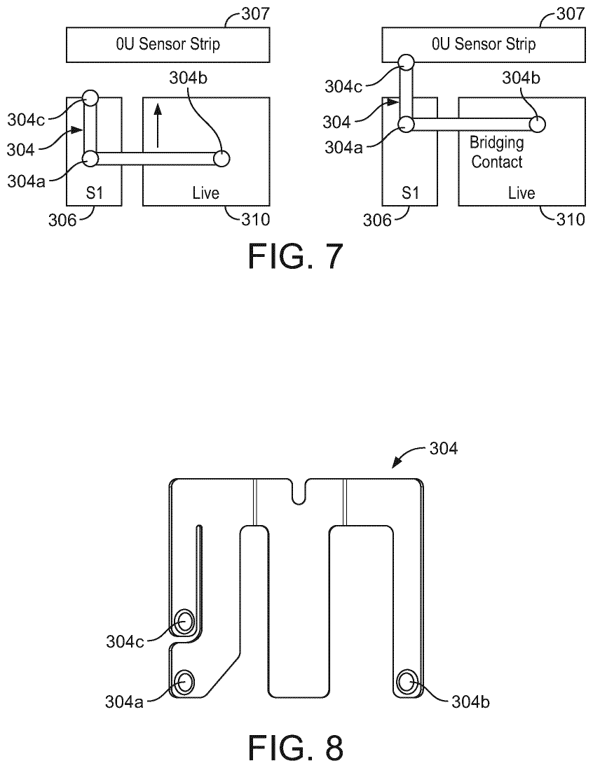

The 0U sensor strip areas 307a-307c are connected together and do not change state during dialling. When the dose delivery button 308 is depressed up to the mechanical stop 320 (0U condition), the bridging contacts 304 electrically connect at least one of the 0U sensor strip areas 307a-307c to a live strip 310, as illustrated by FIG. 7.

Referring again to FIG. 8, each bridging contact 304 has a third point of contact 304c, axially proximal to the two contact points 304a, 304b required for rotational encoding. The third point of contact 304c is designed to contact the 0U sensor strip 307 when the dose delivery button 308 is pressed into the 0U condition.

During dispensation of medicament, the programming component 302 is axially displaced in a distal direction. The 0U sensor strips 307a-307c are advanced toward the bridging contacts 304 which are mounted adjacent the proximal end of the first part 104 of the housing 102. With the dose delivery button pressed into the 0U condition, at least one of the bridging contacts 304 will provide a conductive path between a live strip 310 and a 0U sensor strip 307a-307c, so that the 0U sensor strip 307 is triggered. In other words, the 0U sensor strip 307 is triggered when the third point of contact 304c of the corresponding bridging contact 304 passes a distal edge 330 of the 0U sensor strip 307. Said distal edge 330 defines the switch position 340.

By providing three bridging contacts 304 spaced 120° apart, at least one bridging contact 304 will be in contact with a live strip 310. Therefore, one of the three 0U sensor strips 307a-307c is guaranteed to be triggered when the dose delivery button 308 is pressed into the 0U condition.

The three 0U sensor strips 307a-307c are joined electrically and form a single input to the processor 202. The electrical state of the 0U sensor strips 307a-307c is input to the processor 202, allowing the processor 202 to detect whether the dose delivery button has been pressed into its 0U position. The processor 202 may be further configured to provide a visual or audio indication to the user that the 0U condition has been reached.

The tolerance variation of the 0U sensor strip 307 relative to the mechanical stop 320 means that it cannot be guaranteed that the mechanical stop 320 will be reached at the same time the 0U sensor strip 307 is triggered. This can lead to inaccuracy in the recorded dose. For example, if the 0U sensor strip 307 is triggered before the mechanical stop 320 is reached then a smaller dose will be delivered than is recorded. Conversely, if the mechanical stop 320 is reached before the 0U sensor strip 307 is triggered, then no dose will be recorded.

To prevent the device 100 failing to record a dose, the 0U sensor strip 307 is nominally triggered before the mechanical stop 320 is reached. This is shown schematically in FIG. 10. The bridging contacts 304 are configured to trigger the 0U sensor strip 307 before the mechanical stop 320 is reached in all tolerance conditions. In other words, the bridging contacts 304 are positioned—relative to the 0U sensor strip 307—closer to the proximal end of the device 100 than the mechanical stop 320 by a distance greater than the manufacturing tolerance.

Configuring the device 100 in this way gives rise to the possibility that a user releases pressure from the dose delivery button 308 before the mechanical stop 320 is reached, resulting in a smaller dose being delivered than is recorded. It is important therefore that the user continues to apply pressure to the dose delivery button 308 after the 0U sensor strip 307 is triggered. It is an object of the present disclosure to overcome this problem by providing a delay before an audio or visual cue is given to the user that the dose has been committed. The device 100 of the present disclosure is also required to detect if the dose delivery button 308 has been released before the end of said delay, herein delay period.

The dosage delivery button 308 has an in condition and an out condition. FIG. 9a and FIG. 9b show the dose delivery button in the in condition and the out condition, respectively, relative to bridging contacts 304. The dose delivery button 308 is resiliently biased toward the out condition by a spring (not shown) of the drug delivery mechanism. When a user depresses the dose delivery button 308 during injection, the spring is compressed and the dose delivery button 308 adopts the in condition. Further displacement of the dose delivery button 308 acts to displace the drug delivery mechanism and dispense medicament. Whenever the user releases pressure from the dose delivery button 308 it returns to the out condition. As the dosage programming unit 302 is displaced with the dose delivery button 308, the dosage programming unit 302 also moves between the in and out conditions depending on whether user pressure is applied to the dose delivery button 308.

In the out condition, it is not possible for the bridging contact 304 to make a connection with the 0U sensor strip 307, even in the 0U condition, as shown in FIG. 9a. Therefore, once the user has pressed the dose delivery button 308 into the 0U condition, they must retain pressure on the dose delivery button 308 to maintain the connection between the 0U sensor strip 307 and a live strip 310. Releasing pressure from the dose delivery button 308 will break the connection between the live strip 310 and the 0U sensor strip 307. In the in condition, the third point of contact 304c overlies the 0U sensor strip 307 to make a connection with a live strip 310. However, in the out condition, this connection is not made and the 0U sensor strip 307 is not triggered.

Early release of the dose delivery button 308 is detected in the following way. When a user depresses the dose delivery button 308 into the 0U condition the 0U sensor strip 307 is triggered. The electrical state of the 0U sensor strip 307 is input to the processor 202 which begins to time the delay period. If the user releases pressure from the dose delivery button 308 it will move into the ‘out condition’. This is detected by the processor 202 as a change in the electrical state of the 0U sensor strip 307. If this change of state occurs before the end of the delay period, early release has occurred and the device can flag an error—for example, by generating an error code. If instead the user maintains pressure on the dose delivery button 308, the device will provide the audio or visual cue that dose delivery is complete at the end of the delay period.

The error code is indicative of a deviation between manipulations applied by the user to the device and a predetermined handling scheme. Manipulations applied by the user may be the pressing and releasing of the drug delivery mechanism. The predetermined handling scheme may be pre-programmed into the drug delivery device during manufacture and may comprise one or more thresholds specifying times for which the end of dose switch should remain triggered in order for the associated drug delivery to be considered complete. In this particular example, the error code is indicative of incorrect use of the device. More particularly, the error code indicates that the dose of medicament delivered during use of the device is less than a predetermined dose of medicament. By flagging an error in this way, the user can be prompted to check their dose record or change their dose behaviour.

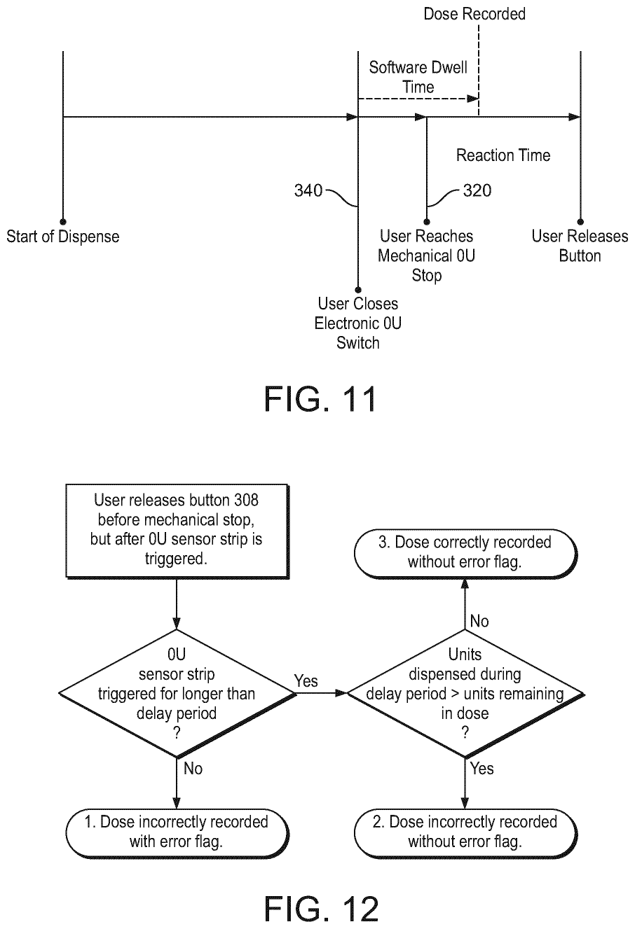

There is a possibility that a user may trigger the 0U sensor strip 307; reach the mechanical stop 320; and then immediately release the dose delivery button 308 before the end of the delay period. In this case, the device as so far described would flag an error, despite the mechanical stop 320 having been reached and the complete dose being delivered. To mitigate this, it is desirable to set the delay period to human reaction time, or slightly below. This will ensure that for the vast majority of cases, the dose will be logged as being fully dispensed before a user can react to reaching the mechanical stop 320 and release the dose delivery button 308. This is shown schematically in FIG. 11. FIG. 11 shows that even in a case where the mechanical stop 320 is reached at the same moment the 0U sensor strip 307 is triggered, the delay period is short enough that a dose is recorded without the device flagging an error.

Whenever the 0U sensor strip 307 is triggered, the encoded dose is recorded and stored in the device's 100 flash memory 205. The encoded dose can be recorded against injection event details, such as the time and date that the medicament was dispensed and/or if an error was flagged.

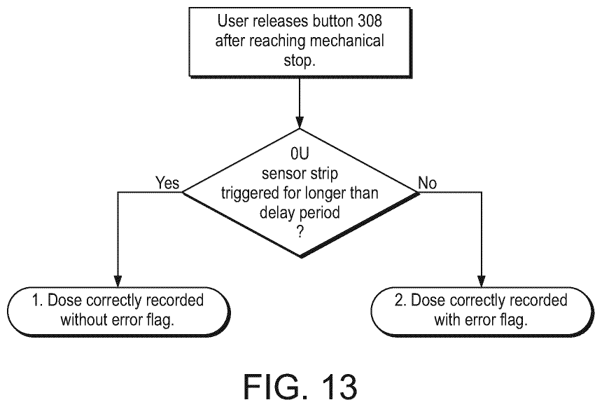

Different outcomes during operation of a device 100 according to the present disclosure will now be explained with reference to flow diagrams FIGS. 12 and 13, which illustrate first and second scenarios, respectively.

### Scenario 1:

FIG. 12 shows the possible outcomes of a user releasing pressure from the dose delivery button 308 before the mechanical stop 320 is reached, but after the 0U sensor strip 307 is triggered. There are three possible outcomes.

### Outcome 1:

If the 0U sensor strip 307 is triggered for less than the delay period—that is to say, if the user releases pressure from the dose delivery button 308 before the end of the delay period—then the dose is recorded, but an error flag is communicated via the device's display 210 to indicate to the user that the encoded dose was recorded with reduced accuracy.

### Outcome 2:

If the 0U sensor strip 307 is triggered for longer than the delay period, but the drug dose delivered is less than the drug dose set and encoded, then the drug dose is recorded without an error flag. Although an error in the recorded dose still exists, it shall be appreciated that the error is significantly less than would be the case if no delay period was used and the user was prompted to release pressure from the dose delivery button 308 even earlier.

Outcome 2 is a possibility if the delay period is set too short to account for the tolerance stack between the 0U sensor strip 307 and the mechanical stop 320. It illustrates the importance of selecting the right delay period so that the audio or visual cue to release the dose delivery button is only given after the mechanical stop 320 has been reached.

### Outcome 3:

The 0U sensor strip 307 is triggered for longer than the delay period and the full amount of the encoded dose is delivered. In this outcome the drug dose is recorded accurately. No error flag is presented to the user and the user's expectation of the drug dose delivered matches the drug dose dispensed.

### Scenario 2:

FIG. 13 shows the possible outcomes of a user releasing pressure from the dose delivery button 308 after the mechanical stop 320 has been reached and the full amount of the encoded dose is delivered. There are two possible outcomes.

### Outcome 1:

The 0U sensor strip 307 is triggered for longer than the delay period. In this outcome the drug dose is recorded accurately. No error flag is presented to the user and the user's expectation of the drug dose delivered matches the drug dose dispensed.

### Outcome 2:

The user reacts to reaching the mechanical stop 320 and releases pressure from the dose delivery button 308 before being given an audio or visual cue to do so by the device 100 and before the end of the delay period. Although the whole dose has been delivered, the dose will be recorded with an error flag, prompting the user to check their dose despite the correct dose being delivered. This further illustrates the importance of selecting the right delay period.

To avoid the second outcome of both scenarios, statistical modelling has been employed to validate a delay period of 0.25 s, slightly less than human reaction time. Two studies were carried out. Each study consisted of a Monte Carlo simulation of 10 million individual events. Each event represented a single dispense by a different user and device.

The first study assumes that the user continues to apply pressure to the dose delivery button 308 for a randomly distributed length of time at the end of each dose, with a normal distribution centred around 5 seconds. The statistical model predicts that for a delay period of 0.25 s, nearly all doses are recorded without an error flag.

The second study assumes that the user releases pressure from the dose delivery button 308 at a uniformly distributed point between where the 0U sensor strip 307 is triggered and the mechanical stop 320 is reached. For this unlikely event, the study predicts that for a delay period of 0.25 s, 99.9% of doses are recorded with less than a 1 unit error, compared to 17.66% in no delay period is implemented.

Although in the above described embodiments the first and second sensor elements 313, 314 of the end of dose switch comprise a conductive strip 307 and a bridging contact 304, respectively, it will be appreciated that other sensor elements may be used without departing from the scope of the present disclosure as, regardless of the type of sensor used, the same problem may arise of a user releasing pressure from the dose delivery button 308 before the mechanical stop 320 is reached, resulting in a smaller dose being delivered than is recorded.

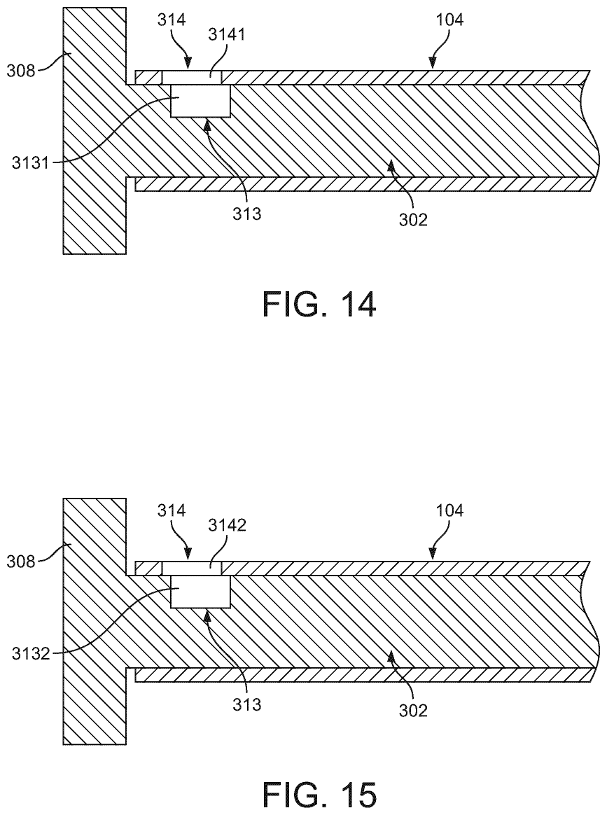

In another embodiment illustrated by FIG. 14, in which like features retain the same reference numbers, the end of dose switch may comprise a Hall Effect sensor. In this embodiment, the first sensor element 313 comprises a semiconductor element 3131 (or alternatively a metal element) and the second sensor element 314 comprises a magnet 3141. The magnet 3141 may be a permanent magnet or an electromagnet. Therefore, when a user depresses the dose delivery button 308 toward the mechanical stop 320 the magnet moves toward the semiconductor element 3131 to generated a Hall voltage. The semiconductor element 3131 is electrically connected to the processor 202 which detects that the magnet 3141 has moved into a switch position 340 when a threshold voltage is met. When the user releases the dose delivery button 308 into the out condition, a second Hall voltage is generated as the magnet 3141 and semiconductor 3131 move out of the switch position 340. The processor 202 is configured to measure the duration of time between the end of dose switch being triggered and the magnet 3141 and semiconductor 3131 moving out of the switch position 340, the processor 202 being further configured to generate an error code indicating a low dose when the measured duration is less than the delay period.

In another embodiment illustrated by FIG. 15, in which like features retain the same reference numbers, the end of dose switch may comprise an induction sensor. In this embodiment, the first sensor element 313 comprises an induction element 3132 and the second sensor element 314 comprises a magnet 3142. The magnet 31421 may be a permanent magnet or an electromagnet. Therefore, when a user depresses the dose delivery button 308 toward the mechanical stop 320 the magnet moves toward the induction element 3132 to induce a current in the induction element 3132. The induction element 3132 is electrically connected to the processor 202 which detects that the magnet 3142 has moved into a switch position 340 when a threshold current is met. When the user releases the dose delivery button 308 into the out condition, a second current is induced as the magnet 3142 and induction element 3132 move out of the switch position 340. The processor 202 is configured to measure the duration of time between the end of dose switch being triggered and the magnet 3141 and induction element 3132 moving out of the switch position 340, the processor 202 being further configured to generate an error code indicating a low dose when the measured duration is less than the delay period.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases.

Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about—4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

1-20. (canceled)

21. A drug delivery device comprising:
a housing having a proximal end and a distal end;
a drug delivery mechanism provided at least partially within the housing and displaceable between a pre-delivery position and a mechanical stop position to dispense medicament from the drug delivery device when a user presses down on a proximal end of the drug delivery mechanism; and
an end of dose switch configured to be triggered prior to the drug delivery mechanism reaching the mechanical stop position, the end of dose switch comprising a first sensor element disposed on the drug delivery mechanism and a second sensor element disposed within the housing;
wherein the end of dose switch is configured to be triggered when relative movement of the first sensor element and the second sensor element causes the first and second sensor elements to move into a switch position, and wherein the drug delivery mechanism is biased in a proximal direction away from the mechanical stop position, so that, when a user ceases to press on the proximal end of the drug delivery mechanism, the first and second sensor elements move out of the switch position;
the drug delivery device further comprising a controller configured to:
record when the end of dose switch is triggered;
measure a duration of time between the end of dose switch being triggered and the first and second sensor elements moving out of the switch position; and
generate an error code indicative of incorrect use of the drug delivery device when a measured duration is less than a predetermined time period.

22. The drug delivery device according to claim 21, wherein the first and second sensor elements move into the switch position when the second sensor element moves over a distal edge of the first sensor element.

23. The drug delivery device according to claim 22, wherein the end of dose switch is configured to be triggered when the first sensor element moves in a distal direction over the switch position.

24. The drug delivery device according to claim 21, wherein the first sensor element comprises a conductive strip on the drug delivery mechanism that is electrically connected to the controller.

25. The drug delivery device according to claim 24, wherein the second sensor element comprises a bridging contact configured to connect the conductive strip to a live strip on the drug delivery mechanism to cause an electrical signal to be transmitted to the controller.

26. The drug delivery device according to claim 25, wherein the bridging contact is formed as a metal pressing and comprises contact points disposed at ends of cantilevered members of the metal pressing, said contact points being configured to contact the live strip and the first sensor element respectively to electrically connect the live strip with the first sensor element.

27. The drug delivery device according to claim 26, wherein the second sensor element comprises two or more bridging contacts spaced around an inner surface of the housing.

28. The drug delivery device according to claim 27, wherein the two or more bridging contacts are provided at the proximal end of the housing.

29. The drug delivery device according to claim 27, wherein the drug delivery mechanism comprises a dose setting component, the dose setting component being rotatable relative to the housing to set a drug dose to be administered; and wherein the first sensor element comprises a series of conductive strips spaced around an outer surface of the dose setting component.

30. The drug delivery device according to claim 29, wherein said dose setting component further comprises conductive dose encoding strips and live strips alternately spaced around the outer surface of the dose setting component, the dose encoding strips each being electrically connected to the controller, and wherein the two or more bridging contacts connect and disconnect the dose encoding strips to the live strips when the dose setting component rotates relative to the housing to transmit electrical signals to the controller and encode a set drug dose.

31. The drug delivery device according to claim 30, wherein the first sensor element comprises a plurality of conductive strips spaced around a proximal end of the dose setting component.

32. The drug delivery device according to claim 31, wherein the plurality of conductive strips of the first sensor element are spaced apart to allow the dose encoding strips to extend in between and into electrical connection with the controller.

33. The drug delivery device according to claim 31, wherein the conductive strips and/or dose encoding strips are printed, plated or etched onto an exterior surface of the dose setting component.

34. The drug delivery device according to claim 30, wherein the controller is configured to record an encoded set drug dose when the end of dose switch is triggered.

35. The drug delivery device according to claim 29, wherein the controller is provided at a proximal end of the dose setting component.

36. The drug delivery device according to claim 21, wherein the predetermined time period is based on human reaction time.

37. The drug delivery device according to claim 21, wherein the predetermined time period is between 0.2 s and 0.4 s.

38. The drug delivery device according to claim 37, wherein the predetermined time period is 0.25 s.

39. The drug delivery device according to claim 21, wherein the drug delivery device is configured to provide a visual and/or audio cue to a user of the drug delivery device to signal that a set drug dose has been administered following expiration of the predetermined time period.

40. The drug delivery device according to claim 21, wherein the drug delivery device is configured to provide a visual and/or audio cue to a user of the drug delivery device to warn a user that a set drug dose may not have been completely administered when the user ceases to press on the proximal end of the drug delivery mechanism prior to an end of the predetermined time period.
